(19) United States
(12) Patent Application Publication
(10) Pub. No.: US 2021/0308404 A1
(43) Pub. Date: Oct. 7, 2021

(54) POSITIVE PRESSURE VENTILATION APPARATUS AND POSITIVE PRESSURE MASK

(71) Applicant: Alphonso Quinones, East Meadow, NY (US)

(72) Inventor: Alphonso Quinones, East Meadow, NY (US)

(21) Appl. No.: 16/839,014

(22) Filed: Apr. 2, 2020

(51) Int. Cl. A61M 16/06; A61M 16/08; A61M 16/10

(52) U.S. Cl. CPC A61M 16/0616; A61M 16/0622; A61M 16/1065; A61M 16/0683; A61M 16/0816; A61M 16/0688

(57) ABSTRACT

A positive pressure ventilation apparatus is provided for non-invasive ventilation of a patient requiring ventilatory care which comprises a positive pressure mask, a positive-pressure source coupled to the positive pressure mask, and an exhalation filter positioned on a gas flow path interconnecting the positive-pressure source and positive pressure mask. The positive pressure mask comprises a mask body configured to cover a patient's mouth and nostrils, the mask body having a patient-contact perimeter, the mask body comprising a flexible concertinaed portion at or adjacent to the patient-contact perimeter to accommodate movement of the patient's face during use, a gas inlet to the mask body configured to be connected to the positive-pressure source, and an adhesive seal positioned at the patient-contact perimeter to secure the mask body to the patient's face and prevent gas leakage during flexion of the concertinaed portion.

### FIELD OF THE TECHNOLOGY

The present disclosure relates generally to apparatuses for non-invasive ventilation of a patient requiring ventilatory care, and more specifically to an enhanced positive pressure mask which is configured to provide non-invasive ventilation without gas leak from the mask when the patient moves their head. In particular, this disclosure relates to a positive pressure mask which can be adhered to the patient's face, as well as having a soft or flexible concertinaed portion which accommodates movement of the patient's head without introducing gas leaks.

### BACKGROUND OF THE INVENTION

Medical professionals caring for patients with respiratory infections, particularly for highly infectious diseases such as the novel coronavirus disease 2019 (COVID-19), are at high risk of contracting the infection.

Aerosol-generating procedures, such as non-invasive ventilation, are of particularly high risk due to mask leaks, typically from around the edge of the mask where the mask meets the face.

Non-invasive ventilation units with an exhalation filter could be used to support acute pulmonary infectious disease patients with respiratory failure. However, the high incidence of mask leakage may result in incomplete filtration, as per Antonio M. Esquinas, S. Egbert Pravinkumar. Noninvasive mechanical ventilation in high-risk pulmonary infections: a clinical review on behalf of the International NIV Network European Respiratory Review 2014 23: 427-438.

This was demonstrated during the Severe Acute Respiratory Syndrome (SARS) pandemic of November 2002, in which non-invasive ventilation created aerosol generation to allow airborne transmission of the virus. There are many case reports of considerable SARS transmission risk with the use of non-invasive ventilators to many patients over extended distances, as per Li Y, Huang X, Yu I T, Wong T W, Qian H. Role of air distribution in SARS transmission during the largest nosocomial outbreak in Hong Kong. Indoor Air 2005; 15: 83-95. Poorly positioned masks result in leaked gas at the masks, causing aerosol dispersion and increasing the risk to medical professionals.

Due to the significant risk of infection to the medical practitioner providing care for a patient with a highly contagious respiratory disease such as COVID-19, mechanical ventilation is the primary mechanism for care, despite the risk to the patient of an invasive intervention.

### SUMMARY OF THE INVENTION

According to an aspect of the invention, there is provided a positive pressure ventilation apparatus for non-invasive ventilation of a patient requiring ventilatory care, the positive pressure ventilation apparatus comprising: a positive pressure mask; a positive-pressure source coupled to the positive pressure mask; and an exhalation filter positioned on a gas flow path to the positive pressure mask; the positive pressure mask comprising: a mask body configured to cover a patient's mouth and nostrils, the mask body having a patient-contact perimeter, the mask body comprising a flexible concertinaed portion at or adjacent to the patient-contact perimeter to accommodate movement of the patient's face during use; a gas inlet to the mask body configured to be connected to the positive-pressure source, the gas inlet comprising a moveable connector for a gas conduit; and an adhesive seal positioned at the patient-contact perimeter to secure the mask body to the patient's face and prevent gas leakage during flexion of the concertinaed portion.

The advantages of the present invention are that there is a significantly improved gas sealing which is resistant to patient movements and other displacements which would otherwise result in aerosol escape pathways from behind the mask which could result in cross-infection.

Preferably, the patient-contact perimeter may comprise a cushioned portion.

Optionally, the cushioned portion may comprise a gel cushion.

In one preferred embodiment, the mask body may be at least in part light-transmissible.

The positive pressure ventilation apparatus may further comprise a removable tab over the adhesive seal.

The positive pressure ventilation apparatus may preferably further comprise a strap for engaging with the patient's head to hold the positive pressure mask in position.

Optionally, the moveable connector may comprise an elbow swivel assembly.

Preferably, the flexible concertinaed portion may be continuous around the mask body to permit uniform flexion.

There may be a uniform distance between the flexible concertinaed portion and the patient-contact perimeter around at least a majority of the mask body, and more preferably around the entire mask body.

According to another aspect of the invention, there is provided a positive pressure mask comprising: a mask body configured to cover a patient's mouth and nostrils, the mask body having a patient-contact perimeter, the mask body comprising an at least in part flexible portion at or adjacent to the patient-contact perimeter to accommodate movement of the patient's face during use; a gas inlet to the mask body configured to be connected to a positive-pressure source; and an adhesive seal positioned at the patient-contact perimeter to secure the mask body to the patient's face.

Optionally, the patient-contact perimeter may comprise a cushioned portion.

Preferably, the cushioned portion may comprise a gel cushion.

The positive pressure mask may further comprise a removable tab over the adhesive seal.

Optionally, the gas inlet may comprise an elbow swivel assembly.

The at least in part flexible portion may be a flexible concertinaed portion of the mask body to accommodate movement of the patient's face during use.

According to another aspect of the invention, there is provided a positive pressure mask comprising: a mask body configured to cover a patient's mouth and nostrils, the mask body having a patient-contact perimeter; and a gas inlet to the mask body configured to be connected to a positive-pressure source; the mask body comprising a flexible concertinaed portion at or adjacent to the patient-contact perimeter to accommodate movement of the patient's face during use.

The positive pressure mask may further comprise an adhesive at or adjacent to the patient-contact perimeter.

Optionally, the mask body may be formed from a flexible plastics material.

The flexible concertinaed portion may be continuous around the mask body to permit uniform flexion.

Preferably, there may be a uniform distance between the flexible concertinaed portion and the patient-contact perimeter around at least a majority of, and preferably the entire, mask body.

According to another aspect of the disclosure, there is provided a method for non-invasive ventilating a patient requiring ventilatory care, the method comprising the steps of: a providing a positive pressure ventilation apparatus comprising: a positive pressure mask; a positive-pressure source coupled to the positive pressure mask; and an exhalation filter positioned on a gas flow path interconnecting the positive-pressure source and positive pressure mask; the positive pressure mask comprising: a mask body configured to cover a patient's mouth and nostrils, the mask body having a patient-contact perimeter, the mask body comprising a flexible concertinaed portion at or adjacent to the patient-contact perimeter; a gas inlet to the mask body configured to be connected to the positive-pressure source; and an adhesive seal positioned at the patient-contact perimeter; b] engaging the positive pressure mask over the patient's face by connection of the adhesive seal to secure the mask body to the patient's face and thereby prevent gas leakage during flexion of the concertinaed portion; and c] activating the positive-pressure source to ventilate the patient.

According to another aspect of the disclosure, there is provided a positive pressure mask comprising: a mask body configured to cover a patient's mouth and nostrils, the mask body having a patient-contact perimeter; a gas inlet to the mask body configured to be connected to a positive-pressure source; and an adhesive seal positioned at the patient-contact perimeter to secure the mask body to the patient's face.

Other aspects, as well as features and advantages of various aspects of the disclosed subject matter will become apparent to those of ordinary skill in the art through consideration of the ensuing description, the accompanying drawings, and the appended claims.

### BRIEF DESCRIPTION OF THE DRAWINGS

In the drawings:

FIG. 1 shows a patient wearing an indicative embodiment of a positive pressure mask in accordance with one aspect of the invention, from the front, with an inset showing the concertinaed portion structure of the mask body;

FIG. 2 shows the patient wearing the positive pressure mask of FIG. 1, from the side;

FIG. 3 shows the positive pressure mask of FIG. 1 from the patient-facing direction; and

FIG. 4 shows a positive pressure ventilation apparatus in accordance with the invention.

### DETAILED DESCRIPTION OF PREFERRED EMBODIMENTS

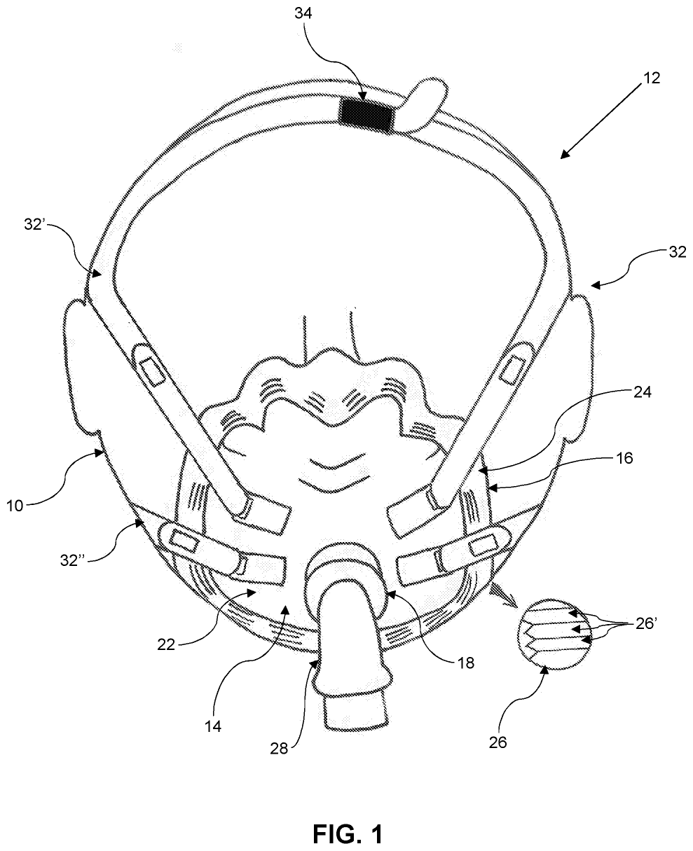

Referring to FIG. 1, there is illustrated a patient 10 wearing a positive pressure mask 12 in accordance with the present disclosure.

The positive pressure mask 12 comprises a mask body 14 which is receivable over the patient's mouth and nose, so as to cover the mouth and nostrils. The mask body 14 may be formed from a flexible material, such as polyvinylchloride or silicone, and therefore may be capable of conforming to the contours of the patient's face at or adjacent to a patient-contact perimeter 16 thereof. However, the mask body 14 could be contoured and dimensioned so as to fit around a predetermined dimension and shape of a patient's face. In such a case, a more rigid mask body 14 material could be used.

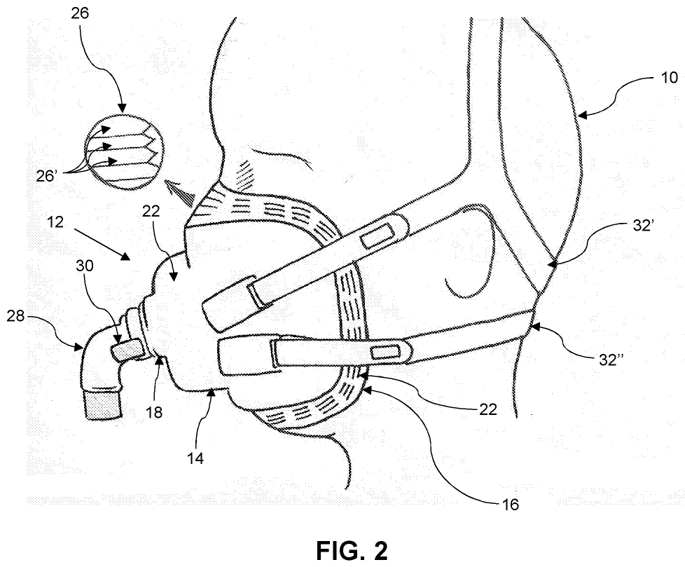

To cover the user's respiratory access, that is, the nostrils and mouth, the mask body 14 is configured to overlie the nose, typically at the bridge of the nose, the cheeks, and under the chin. The patient-contact perimeter 16 therefore forms the points of contact with the patient, as best illustrated in FIG. 2.

The mask body 14, when positioned over the patient's face, forms a ventilation chamber which can be pressurized to urge air or oxygen from a gas inlet 18 of the positive pressure mask 12 into the patient's lungs.

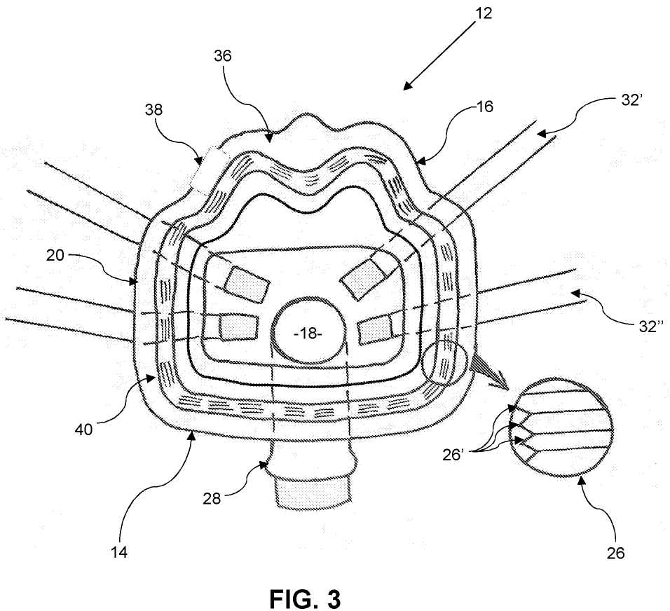

To ensure that the ventilation chamber is sealed to the atmosphere, in order to inhibit release of aerosols and therefore potential contagions for a medical practitioner, the positive pressure mask 12 advantageously may comprise an adhesive seal 20, best seen in FIG. 3, positioned at the patient-contact perimeter to secure the mask body 14 to the patient's face. This prevents or reduces gas leakage from the ventilation chamber.

The mask body 14 may comprise two separate body portions. Firstly, there is a main body portion 22 which forms the bulk of the ventilation chamber, and which may be formed from a rigid or flexible material.

The main body portion 22 preferably has a small projecting profile beyond the patient's mouth and/or nose, in order to minimise wasted volume in the ventilation chamber which could otherwise lead to carbon dioxide rebreathing for the patient. In a preferred embodiment, the main body portion 22 is formed from, or includes at least in part, a light-transmissible material, such as a transparent plastics material. This allows the medical practitioner to visually inspect the patient through the positive pressure mask 12 without needing to remove the positive pressure mask 12.

Secondly, the mask body 14 may preferably include a ridged body portion 24. This ridged body portion 24 may be positioned at or adjacent to the patient-contact perimeter 16 to permit deformation of the mask body 14 during movement of the patient. This ridged body portion 24 comprises a flexible or soft concertinaed portion 26, which allows for compressive or bellows-like motion similar to that of an accordion.

This structure can be best seen in the inset portions of FIGS. 1 and 2. The flexible concertinaed portion 26 comprise at least one concertina ridge 26′, preferably at least two said concertina ridges 26′, and even more preferably at least three concertina ridges 26′. This achieves deformable flexibility at or adjacent to the patient-contact perimeter 16.

The positive pressure mask 12 having such a flexible concertinaed portion 26 is able to accommodate movements of the patient's head within the flexion of the concertina ridges, despite the lag or tension experienced by the mask body 14 from the gas inlet 18.

To further limit the pull on the mask body 14 at the gas inlet 18, the gas inlet 18 preferably comprises a movable joint or connector, such as an elbow swivel assembly 28. This permits rotation around the mask body 14 which may counteract the deformation of the flexible concertinaed portion 26 without increasing the forces thereon. The right-angled or near-right-angled elbow joint of the elbow swivel assembly 28 is particularly appropriate for a prone patient 10. The elbow swivel assembly 28 could be provided so as to be integrally formed with the mask body 14, or could be a removable or releasably engagable component of the positive pressure mask 12, thereby allowing for a more modular construction to be achieved. A releasable connector 30 is illustrated in FIG. 2 in the form of a quick-release snap-fit connector. However, alternative connectors could be provided, such as detent fit, or screw-threaded engagement, and alternative connection mechanisms will be apparent to the skilled person.

To improve the positioning of the positive pressure mask 12 on the patient 10, as well as limiting the propensity for de-adhesion of the adhesive seal 20 over time, a strap 32 for engaging with the patient's head may be provided.

An upper strap 32′ may be provided, which engages from an upper portion of the mask body 14, which connects to the upper portion of the patient's head, typically above the ears. A bifurcated head strap is provided to secure to both the upper and lower occipital areas of the patient's head. A lower strap 32″ may also be provided, which engages from a lower portion of the mask body 14 below the level of the ears, and around an upper portion of the patient's neck.

An adjustable connector 34 may be provided so that the strap 32 can be used with patients of different sizes. Here, a hook-and-loop type fastener is provided, but alternative latch, buckle, or tied fasteners could readily be implemented by the person skilled in the art.

The upper and lower straps 32′, 32″ may be connected to the mask body via connectors on the front of the mask body 14, preferably on the main body portion 22 so as to avoid interfering with the flexible concertinaed portion 26.

FIG. 3 shows the inside of the positive pressure mask 12. In a pre-use condition, the adhesive seal 20 may be protected by a cover member 36 to prevent drying out of the adhesive. This cover member 36 may be releasably engagable with the adhesive seal 20, for example, by the use of a removable release tab 38 at or adjacent to the patient-contact perimeter 16. This allows the adhesive seal 20 to only become activated when required. Alternative release mechanisms could be considered instead of a tab, such as a perforation, or overhang of the material of the cover member 36.

The gas inlet 18 is positioned so as to be at or adjacent to a mouth position of the patient, so as to reduce the dead space in the ventilation chamber.

To improve a comfort of the positive pressure mask 12 for the patient, a cushioned portion may be provided, preferably at or adjacent to the adhesive seal 20. This may be provided as a gel cushion, such as the silica gel cushion 40 illustrated. Given the adhesive seal 20 will be positioned on the patient 10 for a long period of time, this will improve comfort for the patient.

It is preferred that there is uniform flexion around the flexible concertinaed portion 26, since this will permit movement in all directions. To this end, it is preferred that the flexible concertinaed portion 26 be continuous around the mask body to permit said uniform flexion. A uniform distance between the flexible concertinaed portion 26 and the patient-contact perimeter 16 and/or adhesive seal 20 at least a majority of the mask body 14 may also assist in this regard. In FIG. 3, there is a minor change in the distance between the patient-contact perimeter 16 and the flexible concertinaed portion 26 at the bridge of the nose, but this will not have a significant effect on the flexible capabilities of the positive pressure mask 12, since there will be minimal movement at the nose.

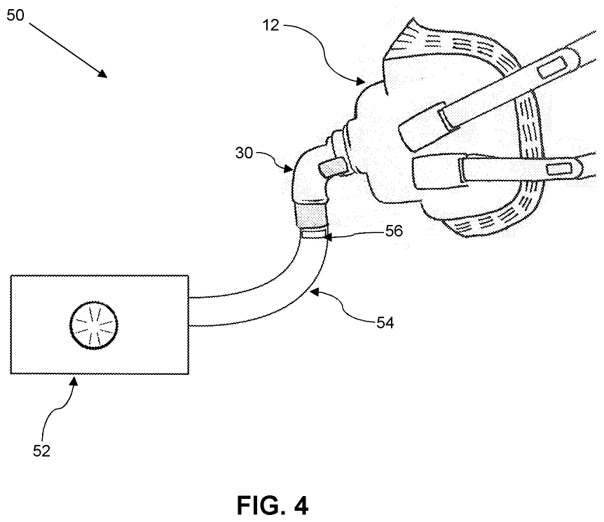

FIG. 4 shows a positive pressure ventilation apparatus, referenced globally at 50, utilising the positive pressure mask 12 as previously described.

The positive pressure mask 12 in connected to a positive-pressure source 52, such as a peristaltic pump used as a ventilator, which enables pressurized gas flow along a gas flow path between the pump and the positive pressure mask 12. The gas flow path is formed by the provision of a connector conduit 54 extending from the positive-pressure source 52 and the positive pressure mask 12, and any appropriate type of gas-tight connector could be used here which is suitable for medical-grade use.

An exhalation filter 56 may advantageously be positioned on a gas flow path, which filters out contagions from the patient's exhaled breath. This may preferably be positioned as close to the positive-pressure mask 12 as is feasible, and is illustrated in FIG. 4 as being directly subsequent to the elbow swivel assembly 28. It will be apparent that the exhalation filter 56 could be provided further along the gas flow path, for example, closer to the positive-pressure source 52, or could indeed be integrally formed with the positive pressure mask 12 or a breathing circuit assembly of the positive-pressure source 52.

The positive pressure ventilation apparatus 50 provides a complete system for non-invasively ventilating a patient 10 in need of ventilatory care. The positive pressure ventilation apparatus 50 could be provided as a kit of parts, or could be provided as a complete set of components.

To use the positive pressure ventilation apparatus 50, the apparatus is assembled, that is, the positive pressure mask 12 is provided, and the positive-pressure source 52 coupled to the positive pressure mask, preferably with the exhalation filter 56 positioned on a gas flow path interconnecting the positive-pressure source 52 and positive pressure mask 12.

The positive pressure mask 12 is then engaged over the patient's face by connection of the adhesive seal 20 to secure the mask body 14 to the patient's face and thereby prevent gas leakage during flexion of the flexible concertinaed portion 26. The positive-pressure source 52 can then be activated to ventilate the patient 10.

While the positive pressure mask is hereto described as having an adhesive seal in combination with a flexible concertinaed portion, it will be apparent to the skilled person that an adhesive seal could be provided with existing positive pressure masks, or indeed with other types of respiratory mask, to achieve improved sealing and therefore reduced cross-infection possibilities. A respiratory mask, and in particular, a positive pressure mask, having an adhesive seal at or adjacent to a patient-contact perimeter of the mask body, is therefore considered to be within the scope of the present invention. This arrangement will be particularly effective in combination with a cushioned portion at or adjacent to the adhesive seal.

Similarly, a positive pressure mask having a flexible concertinaed portion at or adjacent to the patient-contact perimeter, could be provided in the absence of an adhesive seal. In particular, the head straps of the positive pressure mask could be provided to create a sufficient seal without the need for adhesive on the patient's face. This will be particularly effective if the patient-contact perimeter is formed from a deformable or flexible material so as to tightly contour to the patient's face when the positive pressure mask is applied.

While a flexible concertinaed portion is described, it will be apparent that the ridged structure is akin to a bellows, and could be described as such. Furthermore, while a concertina shape is preferred, alternative constructions could be considered. For example, the mask body could comprise the main body portion, formed of a comparatively rigid or hard material, while the flexible portion at/or adjacent to the patient-contact perimeter may be provided as a softer or more flexible material, without necessarily needing to provide the concertina structure proposed above.

As noted previously, any of the advantageous features of the present invention could be applied to respiratory masks other than positive pressure masks, and such mask arrangements are not excluded from the scope of the present invention.

A flexible transparent or translucent material is preferred for the mask body. However, it will be apparent that other materials could be used without any corresponding deterioration in the gas-tightness, without compromising the function of the positive pressure mask.

It is therefore possible to provide positive pressure equipment for ventilating a patient in a non-invasive manner while significantly reducing the risk of transmission of contagions to, in particular, medical practitioners. The positive-pressure mask uses an adhesive seal to improve the sealing capabilities of the mask to the patient's face, while the flexible concertina portion of the mask allows for limited movement of the mask, particularly under the weight of the connectors to the positive pressure source, without creating gas leakage. This provides improved patient ventilation capabilities without the need for an invasive solution.

The words ‘comprises/comprising’ and the words ‘having/including’ when used herein with reference to the present invention are used to specify the presence of stated features, integers, steps or components, but do not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

The embodiments described above are provided by way of examples only, and various other modifications will be apparent to persons skilled in the field without departing from the scope of the invention as defined herein.

1. A positive pressure ventilation apparatus for non-invasive ventilation of a patient requiring ventilatory care, the positive pressure ventilation apparatus comprising:
a positive pressure mask;
a positive-pressure source coupled to the positive pressure mask; and
an exhalation filter positioned on a gas flow path interconnecting the positive-pressure source and positive pressure mask;
the positive pressure mask comprising:
a mask body configured to cover a patient's mouth and nostrils, the mask body having a patient-contact perimeter, the mask body comprising a flexible concertinaed portion of uniform flexion continuously around the mask body, the flexible concertinaed portion comprising a plurality of ridges, at or adjacent to the patient-contact perimeter to accommodate movement of the patient's face during use, the plurality of ridges stacked perpendicular to the patient's face, such that the plurality of ridges are configured to move away from and toward the patient's face;
a gas inlet to the mask body configured to be connected to the positive-pressure source; and
an adhesive seal positioned at the patient-contact perimeter to secure the mask body to the patient's face and prevent gas leakage during flexion of the concertinaed portion, the adhesive seal being positioned at an outermost portion of the patient contact perimeter, the adhesive seal further comprising a cover member and a removable tab.

2. The positive pressure ventilation apparatus as claimed in claim 1, wherein the patient-contact perimeter comprises a cushioned portion.

3. The positive pressure ventilation apparatus as claimed in claim 2, wherein the cushioned portion comprises a gel cushion.

4. The positive pressure ventilation apparatus as claimed in claim 1, wherein the mask body is at least in part light-transmissible.

5. (canceled)

6. The positive pressure ventilation apparatus as claimed in claim 1, further comprising a strap for engaging with the patient's head to hold the positive pressure mask in position.

7. The positive pressure ventilation apparatus as claimed in claim 1, wherein the gas inlet comprises an elbow swivel assembly.

8. (canceled)

9. The positive pressure ventilation apparatus as claimed in claim 1, wherein there is a uniform distance between the flexible concertinaed portion and the patient-contact perimeter around at least a majority of the mask body.

10. A positive pressure mask comprising:
a mask body configured to cover a patient's mouth and nostrils, the mask body having a patient-contact perimeter, the mask body comprising an at least in part flexible portion of uniform flexion continuously around the mask body, the flexible concertinaed portion comprising a plurality of ridges, at or adjacent to the patient-contact perimeter to accommodate movement of the patient's face during use, the plurality of ridges stacked perpendicular to the patient's face, such that the plurality of ridges are configured to move away from and toward the patient's face;
a gas inlet to the mask body configured to be connected to a positive-pressure source; and
an adhesive seal positioned at the patient-contact perimeter to secure the mask body to the patient's face, the adhesive seal being positioned at an outermost portion of the patient contact perimeter the adhesive seal further comprising a cover member and a removable tab.

11. The positive pressure mask as claimed in claim 10, wherein the patient-contact perimeter comprises a cushioned portion.

12. The positive pressure mask as claimed in claim 11, wherein the cushioned portion comprises a gel cushion.

13. (canceled)

14. The positive pressure mask as claimed in claim 10, wherein the gas inlet comprises an elbow swivel assembly.

15. (canceled)

16. A positive pressure mask comprising:
a mask body configured to cover a patient's mouth and nostrils, the mask body having a patient-contact perimeter;
a gas inlet to the mask body configured to be connected to a positive-pressure source;
the mask body comprising a flexible concertinaed portion at or adjacent to the patient-contact perimeter to accommodate movement of the patient's face during use, the flexible concertinaed portion comprising a plurality of ridges, the plurality of ridges stacked perpendicular to the patient's face, such that the plurality of ridges are configured to move away from and toward the patient's face; and
an adhesive seal positioned at the patient-contact perimeter to secure the mask body to the patient's face, the adhesive seal being positioned at an outermost portion of the patient contact perimeter.

17. (canceled)

18. The positive pressure mask as claimed in claim 16, wherein the mask body is formed from a flexible plastics material.

19. The positive pressure mask as claimed in claim 16, wherein the flexible concertinaed portion is continuous around the mask body to permit uniform flexion.

20. The positive pressure mask as claimed in claim 16, wherein there is a uniform distance between the flexible concertinaed portion and the patient-contact perimeter around at least a majority of the mask body.
